(19) United States
(12) Patent Application Publication
(10) Pub. No.: US 2014/0150311 A1
(43) Pub. Date: Jun. 5, 2014

(54) MEDICAL INFORMATIONAL KIT FOR EMERGENCY RESPONDERS

(71) Applicant: Glenn BACON, Statesville, NC (US)

(72) Inventor: Glenn BACON, Statesville, NC (US)

(21) Appl. No.: 13/705,830

(22) Filed: Dec. 5, 2012

(51) Int. Cl. A61B 19/00

(52) U.S. Cl. CPC A61B 19/44
USPC 40/634

(57) ABSTRACT

The need for medical information to explain a potential patient's medical condition is greatly desired by emergency medical responders. Often the patient is unconscious or confused and therefore unable to communicate clearly with the emergency responder. Thus there is a need for this information in a location most likely accessible to an emergency responder. The medical information kit has the patient's medical information and comprises at least a triplicate form, two sealed containers, one magnet sign, one adhesive label sign and optionally one key-chain fob sign. One copy of the form is placed in a container and placed in the refrigerator/medicine cabinet, to alert an emergency responder. Another copy of the form is placed is the second sealed container and placed in the glove compartment of the auto of the patient. Lastly, the last sheet of the triplicate form is placed in the patient's wallet, next to the driver's license.

### FIELD OF THE INVENTION

The need for medical information to describe a potential patient's medical condition is greatly desired by emergency medical responders. Often the patient is unconscious or confused and therefore unable to communicate with the emergency responder. Thus there is a need for this information in a location most likely to be accessible to an emergency responder. The medical information kit contains the patient's medical info nation and comprises at least a triplicate form, 2 sealed containers, one magnet sign, one adhesive label sign, and one optional key-chain fob sign. The form is completed by the patient and one copy is placed in a container and placed in the refrigerator, a magnet is placed on the door of the refrigerator to alert an emergency responder. Another copy of the form is placed is the second sealed container and placed in the glove compartment of the auto of the patient. The key chain fob sign is secured to the key chain of the patient's vehicle. Lastly, the last sheet of the triplicate form is placed in the patient's wallet, next to the driver's license. Also the adhesive label may be placed on the glove compartment door of the patient's vehicle, or on the patient's driver's license.

### BACKGROUND

Emergency responders are often hampered by a lack of patient information at the most critical time—when responding to an emergency. While there are many medical alert devices on the market, most are digital and may or may not be readable by the emergency responders. Most emergency responders in rural areas have little or no digital readers or connectivity associated with the emergency vehicle. Moreover the digital devices change every few years and so what is popular now will be out of fashion in a few years. Further, most elderly are not computer or digitally savvy and are not capable of working with a digital device, nor keeping the medical information current. Additionally, in times of emergency, digital devices frequently are out-of-service. There may be no WiFi, no cellular service, no conventional service, no means to inform the emergency responders and the medical care facilities what the patient's current medical condition is. And even if cellular service is working, there is a strong likelihood that the service will be overloaded with calls due to bad weather, thus rendering the service unreliable.

Therefore there is a need to provide emergency responders with a patient's medical information in the most reliable, quickest and simplest manner possible. Moreover the need should cover emergencies occurring in the patient's house, in a patient's auto, or in any public place.

### SUMMARY OF THE INVENTION

In the face of all the various problems set forth above, the present invention is a simple solution and gives emergency responders the highest chance of success of finding and reading the medical information of the patient. Therefore it overcomes all of the potential problems set forth above.

In the broadest sense, the present invention is a medical information kit for emergency responders, comprising at least a triplicate form for completion of medical history by a potential patient, at least two marked sealed containers, at least one magnetic sign for a refrigerator, at least one adhesive label sign, and optionally at least one key chain fob sign.

### BRIEF DESCRIPTION OF THE DRAWING

The figures of the drawing are not intended to limit the claims or the scope of the invention, but are merely presented to aid in the understanding of the invention.

FIG. 1 is a perspective view of the medical information kit of the present invention showing a triplicate form, 2 sealed containers, one magnet sign, one adhesive label sign, and one optional key-chain fob sign.

FIG. 2 is a perspective view of the components of the medical information kit.

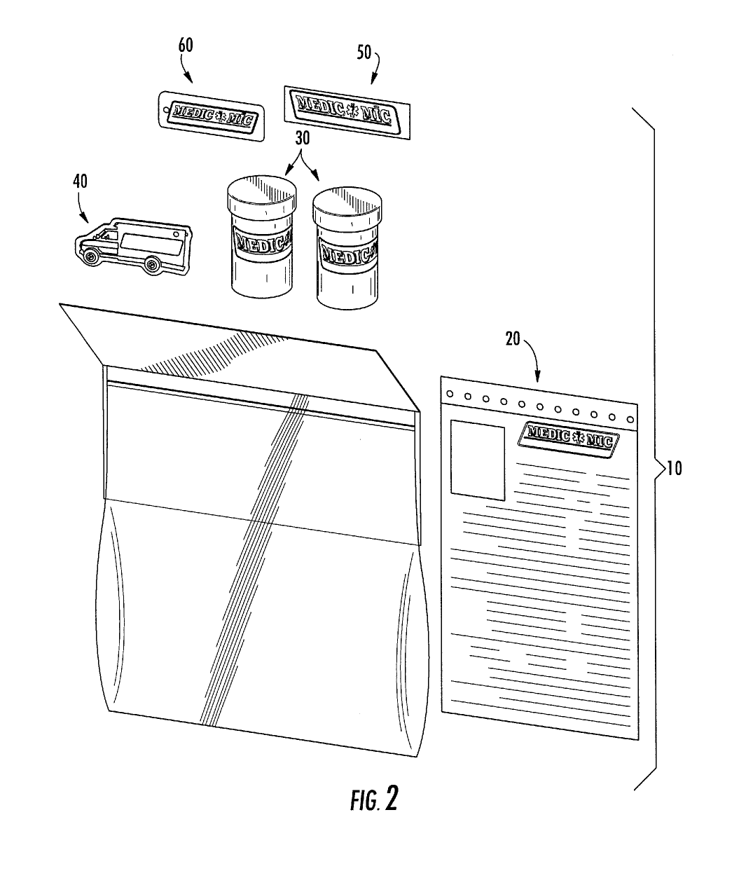

### DESCRIPTION OF THE PREFERRED EMBODIMENTS

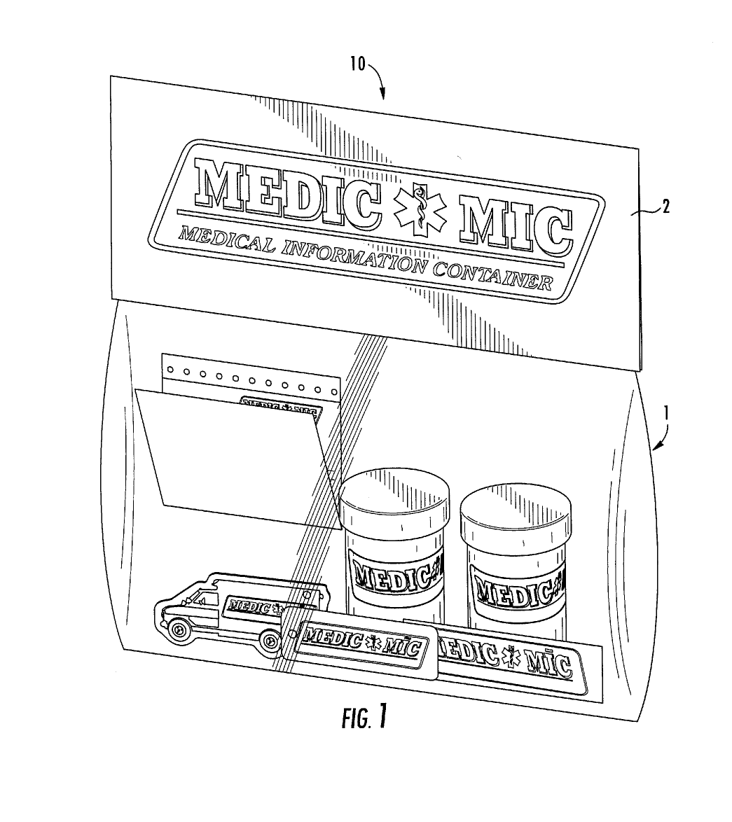

The present invention is a medical information kit tradenamed Medic Mic (pronounced Medic Mike). This kit is desirably printed in red letters with a red logo that will attract emergency medical responders. The contents of the kit are contained in a plastic transparent bag 1 with the tradename card 2 sealing the kit, as shown in FIG. 1. The kit 10 shown in the drawings may be any trade name or nickname, but for purposes of illustration, the tradename is Medic Mic. The kit contains at least a triplicate form 20 containing medical history or information prepared by the patient to cover emergencies in the patient's house, in the patient's auto, and in public places. Should the patient have multiple autos or multiple houses, the form may require additional sheets, or an additional triplicate form, as well as other items of the kit. The backside of the form describes the medical information kit as well as a step by step description of how to use the kit. The front side of the form seeks the patient's name, address, date of birth, a list of the patient's doctors and their phone numbers, the current list of frequent medication and dosage amount, blood type, insurance company name and phone number and account number, Medicaid and Medicare numbers, as well as any known allergies—especially to medication, current medical condition as well as past medical condition, blood pressure, and emergency contact info. Optional information such as hospital preference, pharmacy preference, medical equipment, and replacement body parts such as replacement knee(s) and hip(s), metal plates or screws, replacement heart, kidney or other body parts, diabetes monitor and/or insulin pump, and pace maker can also be recorded on the form.

The form is to be completed by the patient or a trusted assistant upon receiving the medical information kit—and before any medical emergency arises. Moreover it is essential that the patient keep the form updated. Therefore additional triplicate forms can be obtained when updating is needed. Once the form is complete, or after each updating, the patient or the assistant places one copy of the form in a marked sealed container 30 and places it is a conspicuous place in the refrigerator and/or in a medicine cabinet. If it is placed in the refrigerator, the magnetic alert sign 40 should be attached in a prominent place on the refrigerator door so that emergence responders will spot it. Most emergency responders, when responding to an emergency in a home, will check the refrigerator where certain medicines that must be maintained cool are kept, and check the medicine cabinet. On the medicine cabinet, the patient is to place an adhesive alert sign 50. Now it is highly likely the emergency responders will see the alerts and find the information in the marked sealed containers, read it and forward the info to the medical facility.

A second sheet of the triplicate form is placed in the second marked sealed container 30, depending on need, and placed in the glove compartment of the patient's auto or vehicle. Likewise, the patient's key ring should contain the alert key chain fob 60. Optionally an alert adhesive sign 50 can be placed on the door of the glove compartment. In the event of an auto or vehicle event involving a patient, one of the first things the emergency responders do is turn off and remove the keys of the auto, even if the engine is no longer running, to prevent possible electrical shock, fire, and/or a confused patient from trying to start the auto. Thus it is highly likely the responder will see the medical alert key chain fob or the alert adhesive sign on the glove compartment, recover the marked sealed container, and read the medical information on the second (or higher) sheet, and share the information with the medical facility.

The third sheet of the triplicate form is placed in the patient's wallet adjacent the driver's license. Emergency responders always look at the driver's license of an unconscious or confused victim. In that location they are likely to see the third sheet and read the medical information on the third sheet, and share the information with the medical facility.

Although the kit, in the broadest sense, comprises a triplicate form 20, extra form sheets may be needed. Although the kit also calls for 2 sealed containers 30, extra sealed containers may be necessary. Although the kit calls for a magnetic alert sign 40, extra magnetic signs may be desirable. And although the kit calls for an alert adhesive sign 50, extra alert adhesive signs may be warranted. Lastly, although the kit calls for an optional key chain sign fob 60, extra key chain fobs may be wanted. For example, if the patient has medicine in both the refrigerator and medicine cabinet, the patient may need extra form sheets, extra sealed containers and an extra adhesive label to place on the medicine cabinet and the glove compartment, both of which contain the marked sealed containers. If the patent has two autos, there may be a need for an extra informational form sheet, an extra sealed container, and optionally an extra key chain fob sign. If the patient has two homes, the kit expands to meet the needs of the patient, depending on how many refrigerators and medicine cabinets are employed to house the medication. Thus each kit can be customized to an individual patient's needs.

Thus it is apparent that there has been provided, in accordance with the invention an emergency medical information kit The application fully satisfies the objects, aims, and advantages set forth above. While the invention has been described in conjunction with specific embodiments thereof, it is evident that many alternatives, modifications, and variations will be apparent to those skilled in the art in light of the foregoing description. Accordingly it is intended to embrace all such alternatives, modifications and variations as fall within the spirit and broad scope of the appended claims.

What is claimed is:

1) A medical information kit for emergency responders, comprising at least a triplicate form seeking medical history or medical data from a potential patient, two sealed containers, a magnetic sign for a refrigerator, an adhesive label sign and optionally a key chain fob sign.

2) The medical kit of claim 1, wherein said at least a triplicate form seeks the name, address, date of birth, a list of the potential patients doctors and their phone numbers, the current list of medication and dosage amount, blood type, insurance company, Medicaid and Medicare numbers, as well as any known allergies.

3) The medical kit of claim 2, wherein said form also seeks current medical condition as well as past medical condition, blood pressure, any cholesterol medication, and emergency contact info.

4) The medical kit of claim 1, wherein said two sealed containers are of a size and shape to easily hold at least one sheet of said at least a triplicate form.

5) The medical kit of claim 4, wherein said containers have indicia thereon to attract attention of the responders.

6) The medical kit of claim 1, wherein said magnetic sign, key chain sign and adhesive label sign have indicia thereon to alert potential emergency responders to medical information of the potential patient.

7) The medical kit of claim 6, wherein said indicia is red-large-printed text.

8) The medical kit of claim 1, further including 2 adhesive label signs.
